(19) United States
(12) Patent Application Publication
(10) Pub. No.: US 2004/0015119 A1
(43) Pub. Date: Jan. 22, 2004

(54) Blood disinfection method and system

(76) Inventor: Tomomi Takashima, Tokyo (JP)

Correspondence Address: NOTARO AND MICHALOS, 100 DUTCH HILL ROAD, SUITE 110, ORANGEBURG, NY, 10962-2100, US

(21) Appl. No.: 10/417,649

(22) Filed: Apr. 17, 2003

(30) Foreign Application Priority Data
Apr. 24, 2002 (JP) 2002-122289

(51) Int. Cl. A61M 37/00; A61F 7/00

(52) U.S. Cl. 604/6.08; 607/106

(57) ABSTRACT

This invention relates to a blood disinfection method to disinfect retroviruses and other viruses by repeating the following procedures: extracting blood infected with retroviruses such as HIV, HBV, HCV and other viruses from a patient's body, irradiating a YAG laser beam externally on it via a reflection mirror, and returning it to the patient. A disinfection system essentially consists of a low temperature room to lower temperature of a patient in this system, a blood circulation system to circulate blood by extracting blood of the patient from his or her body and returning it, and a laser beam irradiation means to irradiate a laser beam on his or her blood circulating in this blood circulation system.

### BACKGROUND OF THE INVENTION

1. Field of the Invention

This invention relates to a blood disinfection method and system by using a laser beam to disinfect retroviruses such as HIV (AIDS virus), HAV (hepatitis A virus), HBV (hepatitis B virus), HCV (hepatitis C virus) and other viruses in blood circulating through a patient's body.

2. Description of Related Art

Conventional treatment methods for patients infected with retroviruses such as HIV, HAV, HBV, HCV and others are medicament and blood heating with temperature to the degree of not destroying constituents of blood. However, there are no extremely effective medicaments, and there are always side effects on them. Furthermore, just heating blood with temperature to such degree does not destroy these retroviruses. Therefore, no effective disinfection method or system to disinfect the above-mentioned retroviruses with no side effects has been invented.

### SUMMARY OF THE INVENTION

Through many experiments, this inventor invented that irradiation of a YAG laser beam would destroy retroviruses such as HIV, HAV, HBV, and HCV that are not covalently bound on the surface of leukocytes.

An object of this invention is to provide an effective method and a system for disinfection of blood infected with retroviruses such as HIV, HBV, HCV, and other viruses with no side effects by medicaments

To attain this object, blood infected with retroviruses such as HIV, HBV, HCV and other viruses is extracted from a patient's body, and this blood returns to the patient after being irradiated with a YAG laser beam outside a patient's body via a reflection mirror. Repeating this procedure annihilates all retroviruses and other viruses in blood.

Furthermore, by lowering the patient's temperature until he or she is half unconscious and slowing blood circulation, the effect of laser beam irradiation is increased.

The system of this invention is composed of a low temperature room as means for lowering temperature of a patient in this room, a blood circulation system as means for extracting all the patient's blood from his or her body and returning it to there, and a laser beam irradiation means for irradiating a laser beam on the blood in this blood circulation system.

This invention, regarding irradiation of a laser beam on all the patient's blood, is to irradiate the laser beam evenly on blood in a pipe by diffusing the laser beam into a sector form, reflecting it with a reflection mirror, and then irradiating this beam on the blood flowing through the pipe made with materials which permeate lights. A YAG laser beam is used as a laser beam.

### BRIEF DESCRIPTION OF THE DRAWINGS

FIG. 1 is a front view of a blood disinfection system relating to this invention;

FIG. 2 is a side view of this blood disinfection system relating to this invention;

FIG. 3 is a block diagram of this blood disinfection system relating to this invention;

FIG. 4 is a view of a blood circulation system of this blood disinfection system relating to this invention; and

FIG. 5 is a view of a laser irradiation means.

### DETAILED DESCRIPTION OF THE PREFERRED EMBODIMENT(S)

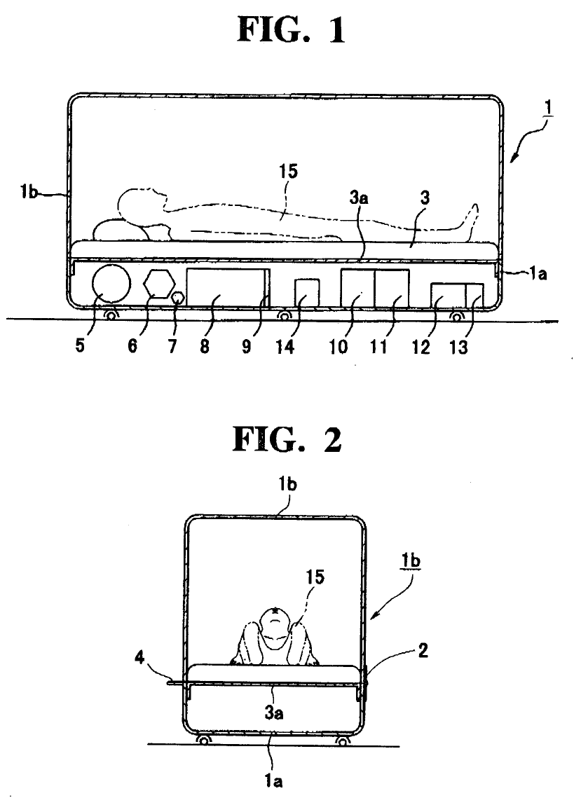

The above-mentioned drawings describe an embodiment of this invention. From FIG. 1 to FIG. 3, a reference number 1 is an air-tightened low temperature room that is composed of a body case 1a, and a hood 1b which opens and shuts by a hinge 2 on the top of the body case 1a. A bed 3 is accommodated therein on a parting board 3a. A reference number 4 is a handle. Packing which is not shown in the drawings is attached on either or both of the top fringe of the body case 1a and/or the bottom fringe of the hood 1b, and this packing seals up both 1a and 1b because of the weight of the hood 1b closed. Fastening tools, e.g., clamps, may be used for avoiding loose sealing. There is no limit for materials of the low temperature room, but synthetic resin, e.g., reinforced plastic, is desirable for them.

As shown in FIG. 1, from the left, e.g., a vacuum pump 5, a low temperature pump 6, a blood return pump 7, a blood disinfection box 8, a filter 9, a 100V-power supply 10, a 200V-power supply 1, a laser beam oscillator 12, a cooling system for this oscillator 13, and a control panel 14 are accommodated in a body case 1a. The order and allocation of the above-mentioned equipments is just an example and not limited to them. Not shown in the drawings, however, an information terminal to collect and manage patient's information such as temperature, heart rates, blood pressure, brain wave and others and a CPU to control them are also accommodated there. The 100V-power supply 10 is the one for the vacuum pump 5, the blood return pump 7, and others. The 200V-power supply 11 is the one for the laser beam oscillator 12. These supplies are divided into two systems, considering electric consumption.

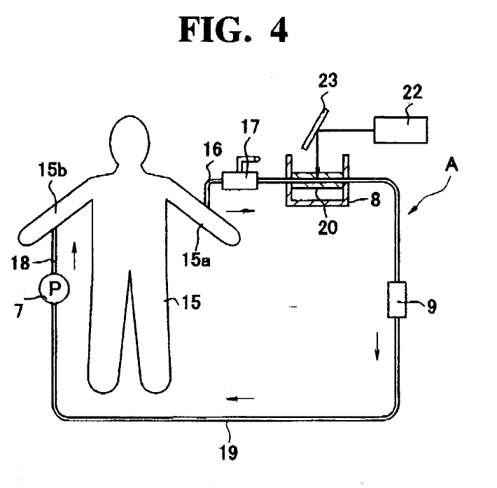

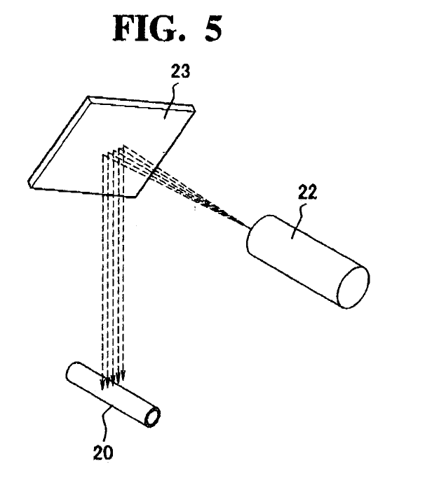

FIG. 4 is a view of a blood circulation system A to extracorporeally circulate blood from the patient's body. A blood extract tube 16 is connected to a not-shown blood vessel in an arm (left) 15a of a patient 15, and a blood return tube 18 is connected to a blood vessel of the other arm (right) 15b of him or her by extending the tube 16. A valve 17 is connected to the blood extract tube 16, and the blood return pump 7 is connected to a blood return tube 18. All the patient's blood is extracted through the blood extract tube 16, passes through a circulation tube 19, and returns to a body through the blood return tube 18. A disinfection pipe 20 with light permeable state accomodated in the laser beam irradiation means is connected to the circulation tube 19, and a laser beam is irradiated from outside of the disinfection pipe 20.

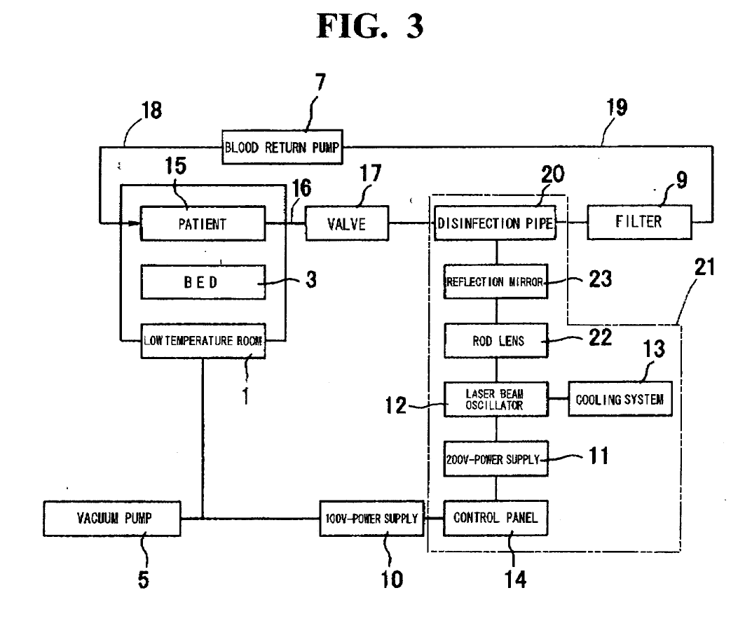

As shown in FIG. 3, the reflection mirror 23 connected to the laser beam irradiation means 21 which is comprised of a 200V-power supply 11, a laser beam oscillator 12, a lod lens 22, and a reflection mirror 23 reflects e.g., YAG laser beam that is diffused into a sector form by a rod lens 22 as shown in FIG. 5, and blood flowing through the disinfection pipe 20 made with materials which transmit lights is irradiated from outside this pipe 20. This blood flows through a filter 9 and the blood return pump 7, and then returns to the patient's 15 body. This blood return pump 7 can be interposed at the upstream of the filter 9.

Here is a case of treatment for a patient infected with retroviruses such as HIV, HCV, and others by using the blood disinfection system according to this invention. As shown in the drawings, after general anesthesia is given as necessary to the patient 15 who is infected with HCV, he or she lies on the back on the bed 3 in the low temperature room 1. The blood extract tube 16 and the blood return tube 18 are respectively connected to e.g., an artery and a vein of an arm of the patient 15. Then, the hood 1b is closed, and air in the low temperature room 1 is evacuated through the vacuum pump 5 so that the patient's 15 temperature reaches at 25, taking about 10 minutes. The patient is half unconscious, and his or her blood velocity at this time is half of that at normal times. His or her blood is extracted from the blood extract tube 16 after opening the valve 17. This blood goes back to the patient through the blood return tube 18 by pumping it with the blood return pump 7. This blood, when flowing through the circulation tube 19, is therefore irradiated with a YAG laser beam from outside the tube.

Blood flowing through a disinfection pipe 20 that is made with crystal glass in the blood disinfection box 8 and connected to the circulation tube 19 is irradiated with the YAG laser beam from outside the pipe. However, just directly radiating the laser beam from the rod lens 22 on the disinfection pipe 20 increases blood temperature so that blood tissues such as erythrocytes, leukocytes, and thrombocytes are destroyed or divided. Therefore, the beam should be diffused into a sector form and irradiated indirectly by using the reflection mirror 23.

It is taken for granted that this circulation and irradiation procedure decreases disinfection effect, so it must be repeated three or four times. The volume of human blood is approximately 15 liters, and blood velocity is down by half due to low body temperature. Therefore, the above-mentioned procedure requires one hour, and three to four hours is required for all procedure to be repeated three or four times.

It is desirable for persons with low blood pressure (100 or below) and high blood pressure (150 or above), persons with a cardiovascular disease, and women during menstruation to avoid this treatment. Temperature and heart rates of a patient must be carefully treated during this treatment.

A laser beam is not always limited to the YAG laser beam. Yb:YAG laser beam is desirable for laser beam, but Nd:YAG is acceptable.

A blood disinfection method and system relating to this invention can disinfect retroviruses such as HIV, HAV, HBV and other viruses in blood.

What is claimed is:

1. A blood disinfection method, comprising the steps of:
extracting blood infected with retroviruses such as HIV, HBV, HCV, and other viruses from a patient's body;
irradiating blood with a YAG laser beam outside the patient's body via a reflection mirror; and
returning the blood to the patient's body,
wherein said steps are repeated plural times.

2. A blood disinfection method according to claim 1, wherein a patient's temperature is lowered until the patient is half unconscious.

3. A blood disinfection system, comprising:
a low temperature room including means for lowering temperature of a patient therein;
a blood circulation system for extracting all of the patient's blood from the patient and returning it to the patient; and
a laser beam irradiation means for irradiating all the patient's blood flowing in said blood circulation system with the laser beam outside of the patient's body.

4. A blood disinfection system according to claim 1, further comprising the steps of:
reflecting said laser beam with a reflection mirror after diffusing said laser beam into a sector form when irradiating said laser beam on the patient's blood; and
irradiating the reflected laser beam on blood flowing through a pipe made with materials which permeate lights.

5. A blood disinfection system according to claim 1, wherein said laser beam is a YAG laser beam.

6. A blood disinfection system according to claim 3, further comprising the steps of:
reflecting said laser beam with a reflection mirror after diffusing said laser beam into a sector form when irradiating said laser beam on the patient's blood; and
irradiating the reflected laser beam on blood flowing through a pipe made with materials which permeate lights.
